(19) United States
(12) Patent Application Publication
(10) Pub. No.: US 2015/0297433 A1
(43) Pub. Date: Oct. 22, 2015

(54) MEDICAL PROCEDURE TABLE WITH INTEGRAL PORTS AND WIRES FOR ELECTROCARDIOGRAPHY

(71) Applicant: Jeanette Owens, Shreveport, LA (US)

(72) Inventor: Jeanette Owens, Shreveport, LA (US)

(21) Appl. No.: 14/691,548

(22) Filed: Apr. 20, 2015

Related U.S. Application Data
(60) Provisional application No. 61/995,684, filed on Apr. 18, 2014.

(51) Int. Cl. A61G 13/00; A61N 1/04; A61G 13/02; A61B 5/0408; A61B 5/1455

(52) U.S. Cl. CPC A61G 13/0018; A61B 5/04085; A61B 5/14552; A61G 13/02; A61N 1/046; A61B 2560/0462; A61B 2562/043; A61B 2562/063; A61B 2562/226; A61B 2562/227; A61B 2562/222

(57) ABSTRACT

A medical procedure table has sensor panels with ports on the top and/or sides of the table for the shorter cables to be attached to the patient and then plugged into the ports of the panels of the table. An equipment panel in the support structure (e.g., pedestal) of the table includes ports for connection to monitoring equipment and drivers. Cables in the table top couple the ports of the sensor panel to corresponding ports of the equipment panel, thus completing a circuit for ECG, SPO₂ monitor, blood pressure cuff, and transducers. Cables may be shielded, unshielded, radiolucent, radiopaque.

### RELATED APPLICATION

This application is a nonprovisional and claims the benefit of priority of U.S. Provisional Application 61/995,684 filed 18 Apr. 2014, the entire contents of which are incorporated herein by this reference and made a part hereof.

### FIELD OF THE INVENTION

This invention relates generally to electrocardiography, and, more particularly, to a table equipped with ports and wiring to facilitate electrocardiography procedures.

### BACKGROUND

Medical equipment such as electrocardiogram equipment utilize a plurality of sensors (e.g., electrodes) to measure physiological parameters. Cables or lead wires connect the sensors to the medical equipment. The sensors typically include adhesive pads. Typically several sensors are applied to a patient to obtain the necessary signals. The raw signals are fed to a processing device such as a computer or electrocardiogram device. By way of example, while performing a cardiac catherization on a patient, a patient's ECG is monitored using sensors connected to monitoring equipment by wire leads.

One serious problem with current equipment is tangling and snagging. ECG leads may be several feet long. Those skilled in the field will readily appreciate that the cables can easily become tangled with each other and with cables for other sensors, and snagged by other equipment and personnel. Detangling consumes time, may delay an urgently needed medical procedure and causes premature failure of cables. Snagging may result in untimely disruption of ECG monitoring.

Another problem is that ECG leads may become contaminated during use due to contact with contaminants such as blood, other bodily fluids, pubic hair, contrast and medicinal preparations. Unfortunately, heretofore, contaminated ECG leads have been reused without adequate sterilization or decontamination.

Another problem is that the integrity of ECG leads may become compromised during repeated use. Bending, stressing and straining an ECG lead may cause structural failure, particularly in the case of radiolucent leads which are quite fragile. Structural defects may prevent transmission of signals or result in spurious signals.

What is lacking in the art is an effective means for managing ECG leads that protects a substantial portion of the leads from tangling, snagging and contamination. The subject invention is directed to overcoming one or more of the problems and fulfilling one or more of the needs as set forth above.

### SUMMARY OF THE INVENTION

To solve one or more of the problems set forth above, in an exemplary implementation of the invention, a medical procedure table has cables installed inside the table top and has sensor interface panels with ports on the top and/or sides of the table for the shorter cables to be attached to the patient and then plugged into the ports of the panels of the table. An equipment interface panel in the support structure (e.g., pedestal) of the table includes ports for connection to monitoring equipment and drivers. The cables in the table top couple the ports of the sensor interface panel to corresponding ports of the equipment interface panel, thus completing the circuit for the ECG, SPO₂ monitor, blood pressure cuff, and transducers. The cables may be shielded or unshielded, radiolucent or radiopaque. This allows the equipment to connect to the support base of the table while the sensors leading to the patient are connected to the sensor interface panels at the table top. Thus the cables (e.g., ECG lead wires) extending from the table top to the patient, may be considerably shorter than those used without such a table, and will not dangle around the table or extend to the monitoring equipment.

An outlet module is provided in the pedestal. The outlets may be isolated ground emergency power receptacles to reduce electrical noise and provide backup generator power during a power outage. The outlets may be hard wired into power supply of the room, on a separate circuit breaker, completely isolated and insulated, to eliminate or reduce 60 cycle noise sensed by the ECG monitoring system or a defibrillator.

The table is tiltable. The end of the table closest to an XRay tech and the feet of the patient includes a foot pad section that folds up into a supportive foot board when the table is set for a tilt procedure. This allows the patient to place their feet on the foot board and stand when the table is tilted at about 70 degrees. As long as there is 5 pounds of force on the foot board, the foot board will not relax out, i.e., return to the unfolded position, even if the table is turned off, malfunctions, or the electricity supply fails. This protects the patient.

### BRIEF DESCRIPTION OF THE DRAWINGS

The foregoing and other aspects, objects, features and advantages of the invention will become better understood with reference to the following description, appended claims, and accompanying drawings, where:

FIG. 1 is a first side view of an exemplary medical table equipped with ECG ports according to principles of the invention; and

FIG. 2 is a second side view of an exemplary medical table equipped with ECG ports according to principles of the invention; and

FIG. 3 is a first perspective view of an exemplary medical table equipped with ECG ports according to principles of the invention; and

FIG. 4 is a second perspective view of an exemplary medical table equipped with ECG ports according to principles of the invention; and

FIG. 5 is a first side view of a tilted exemplary medical table equipped with ECG ports according to principles of the invention; and

FIG. 6 is a second side view of a tilted exemplary medical table equipped with ECG ports according to principles of the invention; and

FIG. 7 is a front view of an exemplary outlet module for a medical table equipped with ECG ports according to principles of the invention; and

FIG. 8 is a perspective view of an exemplary outlet module for a medical table equipped with ECG ports according to principles of the invention; and

FIG. 9 is a front view of an exemplary equipment interface panel for a medical table equipped with ECG ports according to principles of the invention; and

FIG. 10 is a perspective view of an exemplary equipment interface panel for a medical table equipped with ECG ports according to principles of the invention; and

FIG. 11 is a front view of an exemplary sensor interface panel for a medical table equipped with ECG ports according to principles of the invention; and

FIG. 12 is a perspective view of an exemplary sensor interface panel for a medical table equipped with ECG ports according to principles of the invention.

Those skilled in the art will appreciate that the figures are not intended to be drawn to any particular scale; nor are the figures intended to illustrate every embodiment of the invention. The invention is not limited to the exemplary embodiments depicted in the figures or the specific components, configurations, shapes, relative sizes, ornamental aspects or proportions as shown in the figures.

### DETAILED DESCRIPTION

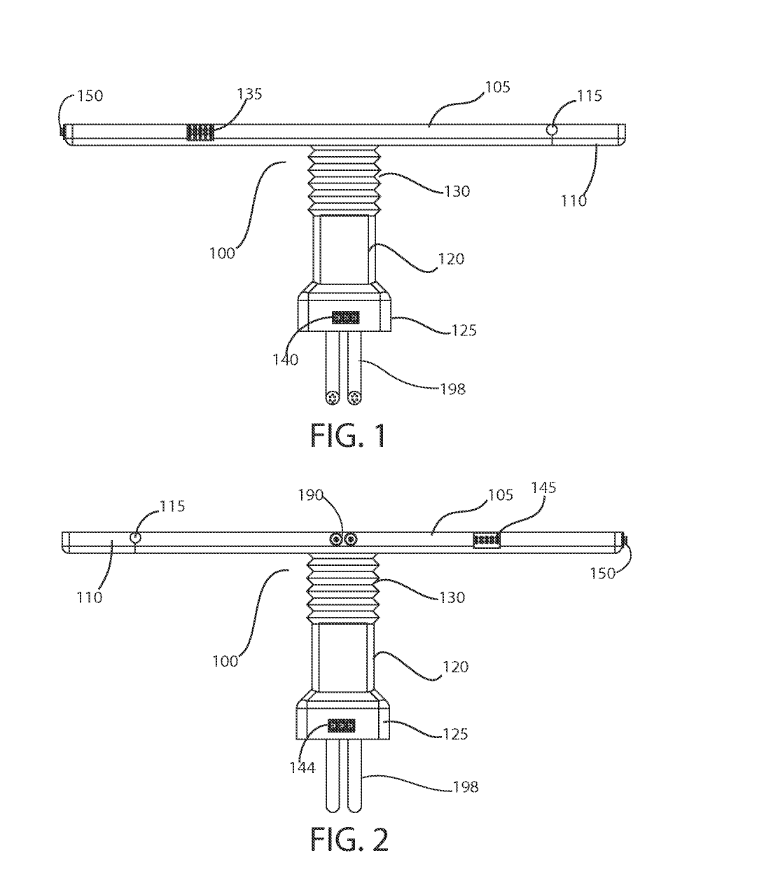

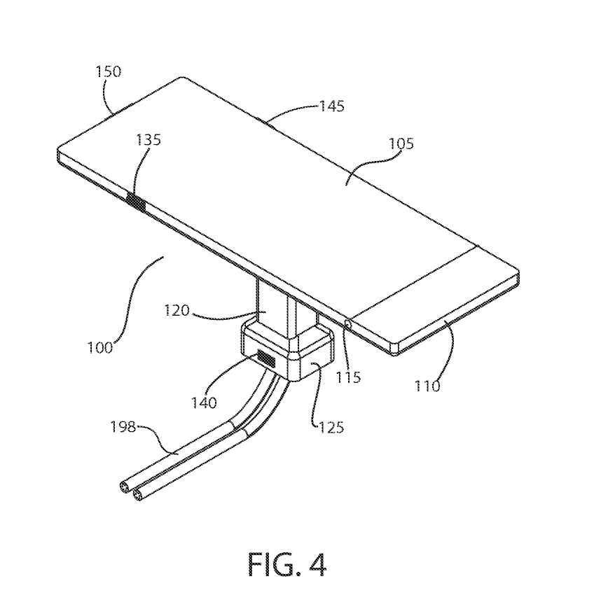

Referring to FIGS. 1 through 4, various views of an exemplary medical table 100 (e.g., Interventional Cardiology X-Ray table or an Interventional Electrophysiology table) equipped with ECG ports according to principles of the invention are provided. The table 100 includes a support pedestal 120 with a base 125 and a flexible bellows 130 that houses height and angular adjustment components. The table also includes a table top 105 suitable for supporting a patient and a pivoting foot pad 110 coupled with a hinge 115. The pedestal 120, particularly the base 125, includes a power outlet module 140, optionally on both sides 140, 144. The table top 105 includes at least one and preferably two or more sensor interface panels 135, 145, 150 and retractable transducer leads 190. Each sensor interface panel 135, 145, 150 is electrically coupled to monitoring equipment via cables 198 extending through channels in the table top 105 and through the pedestal 120. The cables 198 may extend from the pedestal 120 of the table 100 to the monitoring equipment through a trough or subsurface of a medical procedure room floor. The cables 198 may be suitably equipped with connectors at the ends for operably coupling the cables 198 to the equipment.

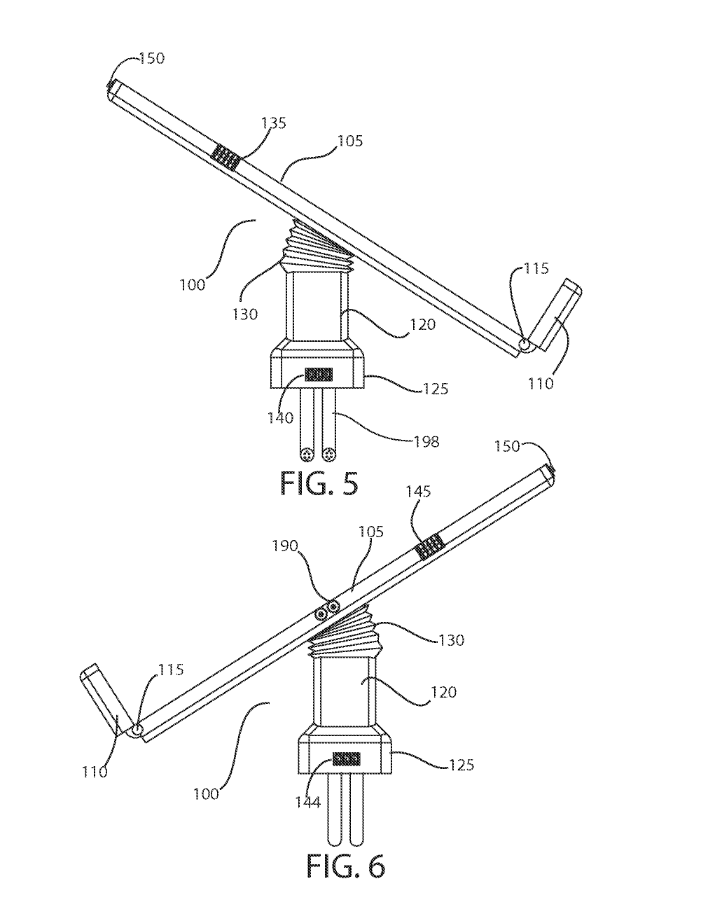

In FIGS. 5 and 6, the table is shown tilted. The end of the table 100 closest to an XRay tech and the feet of the patient includes a foot pad 110 section that folds up into a supportive foot board 110 when the table is set for a tilt procedure. This allows the patient to place their feet on the foot board 110 and stand when the table is tilted at 70 degrees. As long as there is 5 pounds of pressure on the foot board 110, the foot board 110 will not relax out, i.e., return to the unfolded position, even if the table is turned off, malfunctions, or the electricity supply fails. This protects the patient.

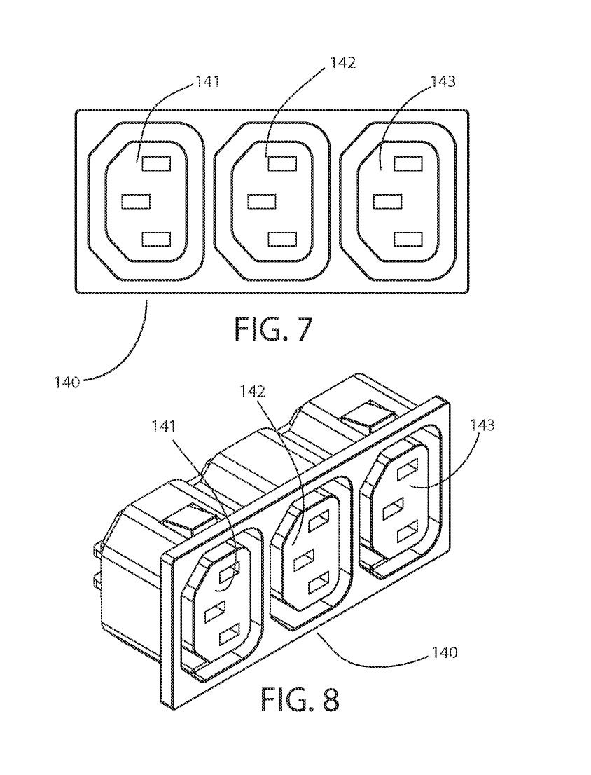

The outlet module 140, as conceptually illustrated in FIGS. 7 and 8, includes a plurality of outlets 141-143 suitable for hospital use. The outlets 141-143 may comprise standard three prong outlets for the country of use, isolated ground receptacles (typically orange in color in the US) to reduce electrical noise, emergency power receptacles (typically red in color in the US) to provide backup generator power during a power outage, or a combination of any of the foregoing. The red outlets may be hard wired into power supply of the room, on a separate circuit breaker, completely isolated and insulated, to eliminate 60 cycle noise sensed by the ECG monitoring system or a defibrillator.

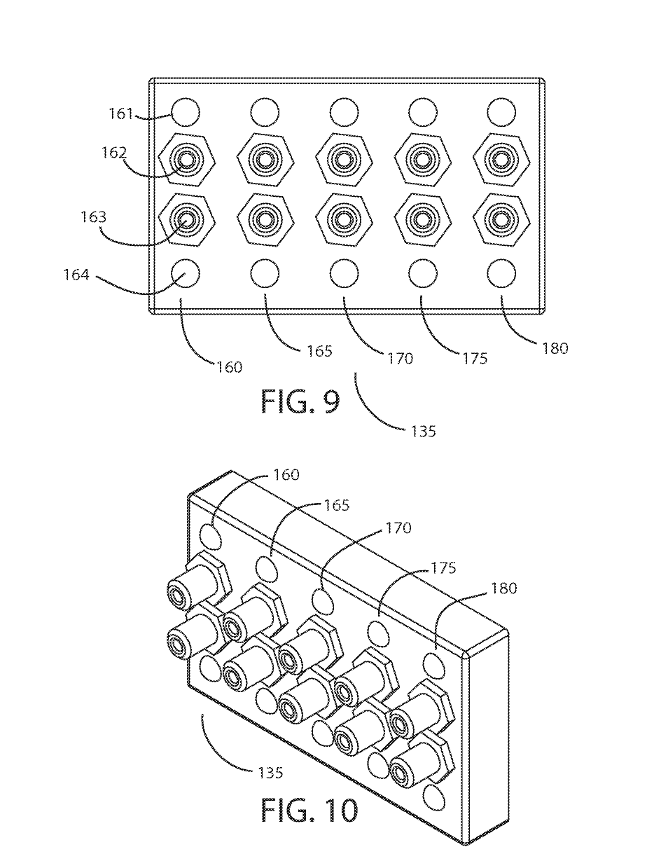

The exemplary sensor interface panel 135, as shown in FIGS. 9 and 10, includes columns 160, 165, 170, 175, 180 of ports 162, 163 and indicators 161, 164. The ports 162, 163. The ports 162, 163 receive electrode leads for electrocardiography. As the sensor interface panel 135 is on the table top 105, short leads will reach the patient. Use of short leads reduces risks of noises that lead to spurious signals, tangling and contamination.

Interface panel 145 is similar to interface panel 135. The panels 135, 145 are located on opposite side of the table 100.

The ports 162, 163 are configured to electrically mate with the lead wires of sensors, e.g., ECG cables with electrodes, SPO₂ monitors. The lead wires may be shielded or non-shielded, radiolucent, or any other suitable lead wire structure. By way of example, and not limitation, Deutsches Institut für Normung (DIN) ports and banana plug ports may be provided for correspondingly equipped ECG lead wires. The distal end of an ECG lead wire, or portion closest to the patient, may include a connector which is adapted to operably connect to the electrode to receive medical signals from the body. The proximal end of the ECG lead wire includes a plug that mates with a port of the sensor interface panel 135. The depicted ports 162, 163 conceptually represent compatible sensor ports and are not limited to any particular type of sensor or coupling. When plugged into a port of the sensor interface panel 135, a sensor, such as an ECG electrode, is electrically coupled to the monitoring equipment via cables 198.

Each column 160, 165, 170, 175, 180 includes one or more indicators 161, 164. The indicators 161, 164 visibly indicate a completed circuit between the electrode and monitoring equipment. The indicators may comprise one or more LEDs or other visible display element. Electrical signals through the wiring in the table top 105 and/or pedestal may be monitored inductively. A microcontroller electrically coupled to the indicators may illuminate the appropriate indicator (e.g., a green LED) when electrical signals are inductively detected. When electrical signals are not detected, the microcontroller may illuminate no indicator, or an indicator (e.g., a red LED) other than the indicator illuminated to indicate a completed working circuit (i.e., other than the green indicator), or may cause a multi-color LED to illuminate a particular color (e.g., red). In this manner, status may be monitored without affecting the communicated signals. Thus, illustratively, a red light indicator at the top of the table shows a disconnect and a green light indicator shows completed active circuitry.

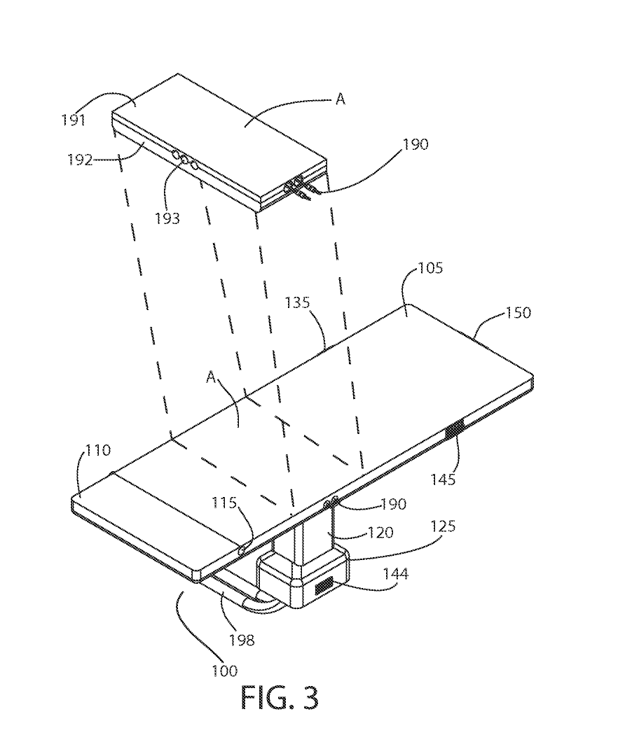

Wiring 198 extending from the pedestal 120 and table top 105 electrically couples each port of the sensor interface panel 135 with monitoring equipment. The wiring may be shielded or non-shielded, radiolucent or radiopaque. In a radiolucent embodiment, the wiring may be comprised of radiolucent electrically conductive material within a radiolucent insulator, such as conductive carbon or conductive carbon monofilament connected to radiolucent (e.g., conductive carbon) contacts in ports using radiolucent electrically conductive adhesive and/or radiolucent crimping (e.g., heat shrinked radilucent plastic tube), with a plastic (e.g., PVC, polypropylene, or polyethylene). In a radiolucent shielded embodiment, a conductive radiolucent coating or sheath (e.g., conductive carbon) is provided on the outer surface of the insulator. The wiring extending from the monitoring equipment through the pedestal 120 and table top 105 may be contained in a flexible sheath. As shown in FIG. 3, the table top 105 may contain one or more compartments or conduits 193 through which the wiring may extend to the sensor interface panel 135, 145, 150. The conduits 193 may be accessible through one or more separable panels or table top components 191, 192. The wiring extending through the table top to the sensor interface panel 135, 145, 150 may also be contained in a sheath.

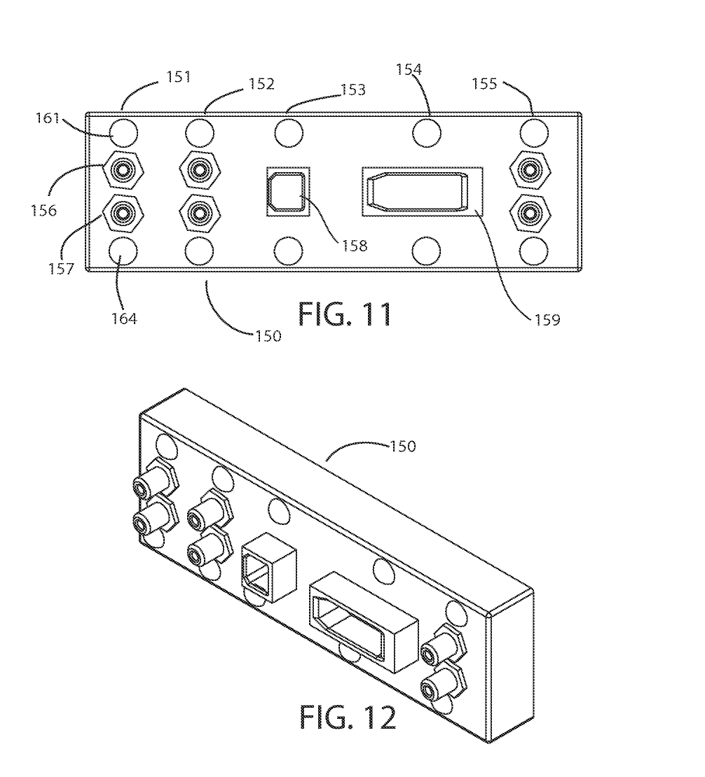

The sensor interface panel 150 at the head of the table 100 includes a plurality of ports/sockets 156, 157, 158, 159 to connect with sensors, devices and equipment, such as, but not limited to ECG electrodes, blood pressure monitoring equipment, pulse oximetry equipment and any other equipment comprising sensors on a patient that produce electrical signals to monitor or measure one or more physiological parameters. The type, number and arrangement of ports on the sensor interface panel 150 are not limited to those shown in FIGS. 11 and 12.

Each column 151, 152, 153, 154, 155 of sensor interface panel 150 includes one or more indicators 161, 164. The indicators 161, 164 visibly indicate a completed circuit between the electrode and monitoring equipment. The indicators may comprise one or more LEDs or other visible display element. Electrical signals through the wiring 198 in the table top 105 and/or pedestal may be monitored inductively. A microcontroller electrically coupled to the indicators may illuminate the appropriate indicator (e.g., a green LED) when electrical signals are inductively detected. When electrical signals are not detected, the microcontroller may illuminate no indicator, or an indicator (e.g., a red LED) other than the indicator illuminated to indicate a completed working circuit (i.e., other than the green indicator), or may cause a multi-color LED to illuminate a particular color (e.g., red). In this manner, status may be monitored without affecting the communicated signals. Thus, illustratively, a red light indicator at the top of the table shows a disconnect and a green light indicator shows completed active circuitry.

The placement of the ECG electrodes on a patient has been established by medical protocols. The most common protocols require the placement of the electrodes in a 3-lead, a 5-lead or a 12-lead configuration. This invention may provide adequate ports for any such configuration, as well as connection of additional sensors and electrodes (e.g., SPO₂ and defibrillator electrodes).

The sensor interface panels 135, 145, 150 may be replaceable. Maintenance may require periodic replacement of the panel and their ports. Such maintenance may involve replacement due to contamination and/or worn contacts. Adaptability may entail replacement. Panels compatible with particular lead wires and monitoring equipment may be utilized.

One or more retractable transducer lead wires 190 or cables are provided. The cables 190 may be withdrawn (pulled out) from the side of the table top 105 to reach a desired portion of the patient. The cables may be retracted into the side of the table top 105 at the end of a procedure. The cables 190 may be retracted on a spring biased reel within the table, a hand reel within the table with a handle accessible outside the table, a motor driven reel within the table, or using another refraction mechanism.

While an exemplary embodiment of the invention has been described, it should be apparent that modifications and variations thereto are possible, all of which fall within the true spirit and scope of the invention. With respect to the above description then, it is to be realized that the optimum relationships for the components and steps of the invention, including variations in order, form, content, function and manner of operation, are deemed readily apparent and obvious to one skilled in the art, and all equivalent relationships to those illustrated in the drawings and described in the specification are intended to be encompassed by the present invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

What is claimed is:

1. A medical table having a table top and a pedestal support, said medical table including:
an equipment interface panel in the pedestal support, said equipment interface panel including a plurality of ports, including at least 3 ECG monitoring ports, each ECG monitoring port being coupled to an ECG monitor;
a sensor interface panel in the table top, said sensor interface panel including a plurality of ports, including at least 3 ECG lead wire ports, each ECG lead wire port being coupled to a lead wire with an ECG electrode,
internal wiring extending from the equipment interface panel to the sensor interface panel, said internal wiring including a wire extending from each port on the equipment interface panel to a corresponding port on the sensor interface panel.

2. A medical table according to claim 1, said internal wiring being shielded.

3. A medical table according to claim 1, said internal wiring being radiolucent.

4. A medical table according to claim 1, further comprising a visible indicator associated with each sensor port, the visible indicator visibly indicating when the port is in use.

5. A medical table according to claim 4, each visible indicator comprising at least one LED associated with each sensor port, the LED illuminating when the port is in use.

6. A medical table according to claim 4, each visible indicator comprising two LEDs, one LED of a first color and another LED of a second color, associated with each sensor port, the first color LED illuminating when the port is in use and the second color LED illuminating when the port is not in use.

7. A medical table according to claim 4, each visible indicator comprising a bi-color LED, having a first color and a second color, associated with each sensor port, the first color of the LED illuminating when the port is in use and the second color of the LED illuminating when the port is not in use.

8. A medical table according to claim 1, further comprising a power supply panel, having a plurality of isolated and grounded power outlets on a separate power supply circuit.

9. A medical table according to claim 1, further comprising a power supply panel, having a plurality of isolated and grounded power outlets on a separate power supply circuit connected to a backup power supply.

10. A medical table according to claim 1, said plurality of ports in the equipment interface panel including at least one SPO₂ monitor port, and said plurality of ports in the sensor interface panel including at least one SPO₂ sensor port, the SPO₂ monitor port being electrically coupled to the SPO₂ sensor port by the internal wiring.

11. A medical table according to claim 1, said plurality of ports in the equipment interface panel including at least one defibrillator port, and said plurality of ports in the sensor interface panel including at least one defibrillator electrode port, the defibrillator port being electrically coupled to the defibrillator electrode port by the internal wiring.

12. A medical table according to claim 1, further comprising a pivoting foot pad attached to the table top by a hinge, said table top being tiltable, and said table top being pivotable from a position in line with the table top to a position orthogonal to the table top, and in the orthogonal position the foot pad providing support surface for a patient when the table is tilted.
